(19) United States
(12) Patent Application Publication
(10) Pub. No.: US 2007/0167628 A1
(43) Pub. Date: Jul. 19, 2007

(54) Thiazole-2-carboxamide derivatives for use as hippar agonists in the treatment of i.a. dyslipidemia

(76) Inventors: Francoise Jeanne Gellibert, Les Ulis (FR); Paul Martres, Les Ulis (FR)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/575,530

(22) PCT Filed: Oct. 7, 2004
(86) PCT No.: PCT/EP04/11386

§ 371 Date: Jan. 4, 2007

(30) Foreign Application Priority Data
Oct. 9, 2003 (GB) 0323702.1

(51) Int. Cl. C07D 471/02

(52) U.S. Cl. 546/122

(57) ABSTRACT

A compound of formula (I) 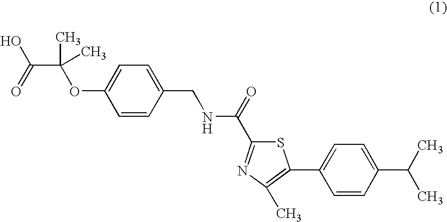 and pharmaceutically acceptable salts, solvates and hydrolysable esters thereof.

The present invention relates to certain novel compounds. In particular, the present invention relates to compounds that activate both the alpha and gamma subtypes of the human peroxisome proliferator activated receptor. The present invention also relates to methods for preparing the compounds and methods for prevention or treatment of PPAR mediated diseases or conditions.

Several independent risk factors have been associated with cardiovascular disease. These include hypertension, increased fibrinogen levels, high levels of triglycerides, elevated LDL cholesterol, elevated total cholesterol, and low levels of HDL cholesterol. HMG CoA reductase inhibitors (“statins”) are useful for treating conditions characterized by high LDL-c levels. It has been shown that lowering LDL-C is not sufficient for reducing the risk of cardiovascular disease in some patients, particularly those with normal LDL-c levels. This population pool is identified by the independent risk factor of low HDL-c. The increased risk of cardiovascular disease associated with low HDL-c levels has not yet been successfully addressed by drug therapy (i.e., currently there are no drugs on the market that are useful for raising HDL-C >40%). (Bisgaier, C. L.; Pape, M. E. Curr. Pharm. Des. 1998, 4, 53-70).

Syndrome X (including metabolic syndrome) is loosely defined as a collection of abnormalities including hyperinsuinlemia, obesity, elevated levels of trigycerides, uric acid, fibrinogen, small dense LDL-c particles, and plasminogen activator inhibitor 1 (PAI-1), and decreased levels of HDL-c.

NIDDM is described as insulin resistance which in turn causes anomalous glucose output and a decrease in glucose uptake by skeletal muscle. These factors eventually lead to impaired glucose tolerance (IGT) and hyperinsulinemia.

Peroxisome Proliferator Activated Receptors (PPARs) are orphan receptors belonging to the steroid/retinoid receptor superfamily of ligand-activated transcription factors. See, for example, Willson, T. M. and Wahli, W., Curr. Opin. Chem. Biol., (1997), Vol. 1, pp 235-241.

Three mammalian Peroxisome Proliferator-Activated Receptors have been isolated and termed PPAR-alpha, PPARgamma, and PPAR-delta (also known as NUC1 or PPAR-beta). These PPARs regulate expression of target genes by binding to DNA sequence elements, termed PPAR response elements (PPRE). To date, PPRE's have been identified in the enhancers of a number of genes encoding proteins that regulate lipid metabolism suggesting that PPARs play a pivotal role in the adipogenic signaling cascade and lipid homeostasis (H. Keller and W. Wahli, Trends Endocrin. Met 291-296, 4 (1993)).

Certain compounds that activate or otherwise interact with one or more of the PPARs have been implicated in the regulation of triglyceride and cholesterol levels in animal models. See, for example, WO 01/40207, WO 01/00603, WO 97/31907, WO 02/46174 (Glaxo Group Ltd et al).

WO 02/096895 describes compounds of the following formula:

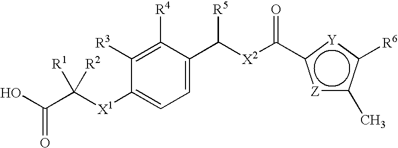

These compounds are described as preferably having PPAR alpha selectivity, useful in the treatment of human PPAR mediated diseases.

According to a first aspect of the invention there is provided a compound of formula (I) or a pharmaceutically acceptable salt, solvate or hydrolysable ester thereof:

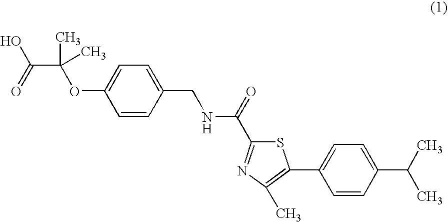

In another aspect, the present invention discloses a method for prevention or treatment of a human PPAR (“hPPAR”) mediated disease or condition comprising administration of a compound of this invention. hPPAR mediated diseases or conditions include dyslipidemia including associated diabetic dyslipidemia and mixed dyslipidemia, syndrome X (as defined in this application this embraces metabolic syndrome), heart failure, obesity, hypercholesteremia, cardiovascular disease including atherosclerosis, arteriosclerosis, and hypertriglyceridemia, type II diabetes mellitus, type I diabetes, insulin resistance, hyperlipidemia, inflammation, opithelial hyperproliferative diseases including eczema and psoriasis and conditions associated with the lining and gut and regulation of appetite and food intake in subjects suffering from disorders such as obesity, bulimia, and anorexia nervosa. In particular, the compounds of this invention may be useful in the treatment and prevention of cardiovascular diseases and conditions including atherosclerosis, arteriosclerosis, hypertriglyceridemia, and mixed dyslipidaemia.

In another aspect, the present invention provides pharmaceutical compositions comprising a compound of the invention, preferably in association with a pharmaceutically acceptable diluent or carrier.

In another aspect, the present invention provides a compound of the invention for use in therapy, and in particular, in human medicine.

In another aspect, the present invention provides the use of a compound of the invention for the manufacture of a medicament for the treatment of a hPPAR mediated disease or condition.

In another aspect, the present invention provides a method of treatment of a patent suffering from a hPPARmediated disease or condition comprising the administration of a compound of the invention.

As used herein, “a compound of the invention” means a compound of formula (I) or a pharmaceutically acceptable salt, solvate, or hydrolyzable ester thereof.

While hydrolyzable esters are included in the scope of this invention, the acids are preferred because the data suggests that while the esters are useful compounds, it may actually be the acids to which they hydrolyze that are the active compounds. Esters that hydrolyze readily can produce the carboxylic acid in the assay conditions or in vivo. Generally the carboxylic acid is active in both the binding and transient transfection assays, while the ester does not usually bind well but is active in the transient transfection assay presumably due to hydrolysis. Preferred hydrolysable esters are C₁₋₆ alkyl esters wherein the alkyl group may be straight chain or branched chain. Methyl or ethyl esters are more preferred.

The compound of formula (I) is a selective dual agonist of PPAR alpha and gamma. As used herein, by “agonist”, or “activating compound”, or “activator”, or the like, is meant those compounds which have a pKi of at least 6.0 preferably at least 7.0 to the relevant PPAR, for example hPPARα in the binding assay described below, and which achieve at least 50% activation of the relevant PPAR relative to the appropriate indicated positive control in the transfection assay described below at concentrations of 10-5 M or less.

As used herein, a “selective dual hPPAR alpha/gamma agonist” is a hPPARalpha/gamma agonist whose EC₅₀ for PPARalpha and PPAR gamma is at least 10 fold lower than its EC₅₀ for PPAR delta. EC₅₀ is defined in the transfection assay described below and is the concentration at which a compound achieves 50% of its maximum activity.

It will be appreciated by those skilled in the art that the compounds of the present invention may also be utilized in the form of a pharmaceutically acceptable salt or solvate thereof. The physiologically acceptable salts of the compounds of formula (I) include conventional salts formed from pharmaceutically acceptable inorganic or organic acids or bases as well as quaternary ammonium acid addition salts. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmoic, malonic, hydroxymaleic, phenylacetic, glutamic, benzoic, salicylic, fumaric, toluenesulfonic, methanesulfonic, naphthalene-2-sulfonic, benzenesulfonic hydroxynaphthoic, hydroiodic, malic, steroic, tannic and the like. Other acids such as oxalic, while not in themselves pharmaceutically acceptable, may be useful in the preparation of salts useful as intermediates in obtaining the compounds of the invention and their pharmaceutically acceptable salts. More specific examples of suitable basic salts include sodium, lithium, potassium, magnesium, aluminium, calcium, zinc, N,N′-dibenzylethylenediamine, chloroprocaine, choline, diethanolamine, ethylenediamine, N-methylglucamine and procaine salts.

The compound of the invention or its pharmaceutically acceptable salts, solvates of hydrolysable esters are conveniently administered in the form of pharmaceutical compositions. Such compositions may conveniently be presented for use in conventional manner in admixture with one or more physiologically acceptable carriers or excipients.

While it is possible that compounds of the present invention may be therapeutically administered as the raw chemical, it is preferable to present the active ingredient as a pharmaceutical composition. The carrier(s) must be “acceptable” in the sense of being compatible with the other ingredients of the formulation and not deleterious to the recipient thereof.

Accordingly, the present invention further provides for a pharmaceutical composition comprising a compound of the invention or a pharmaceutically acceptable salt or solvate thereof together with one or more pharmaceutically acceptable diluent or carrier therefore and, optionally, other therapeutic and/or prophylactic ingredients.

The pharmaceutical compositions include those suitable for oral, parenteral (including subcutaneous e.g. by injection or by depot tablet, intradermal, intrathecal, intramuscular e.g. by depot and intravenous), rectal and topical (including dermal, buccal and sublingual) administration although the most suitable route may depend upon for example the condition and disorder of the recipient. The formulations may conveniently be presented in unit dosage form and may be prepared by any of the methods well known in the art of pharmacy. All methods include the step of bringing into association the compounds (“active ingredient”) with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Pharmaceutical compositions suitable for oral administration may be presented as discrete units such as capsules, cachets or tablets (e.g. chewable tablets in particular for paediatric administration) each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a other conventional excipients such as binding agents, (for example, syrup, acacia, gelatin, sorbitol, tragacanth, mucilage of starch or polyvinylpyrrolidone), fillers (for example, lactose, sugar, microcrystalline cellulose, maize-starch, calcium phosphate or sorbitol), lubricants (for example, magnesium stearate, stearic acid, talc, polyethylene glycol or silica), disintegrants (for example, potato starch or sodium starch glycollate) or wetting agents, such as sodium lauryl sulfate. Moulded tablets may be made by moulding in a suitable machine a mixture of the powdered compound moistened with an inert liquid diluent. The tablets may optionally be coated or scored and may be formulated so as to provide slow or controlled release of the active ingredient therein. The tablets may be coated according to methods well-known in the art.

Alternatively, the compounds of the present invention may be incorporated into oral liquid preparations such as aqueous or oily suspensions, solutions, emulsions, syrups or elixirs, for example. Moreover, formulations containing these compounds may be presented as a dry product for constitution with water or other suitable vehicle before use. Such liquid preparations may contain conventional additives such as suspending agents such as sorbitol syrup, methyl cellulose, glucose/sugar syrup, gelatin, hydroxyethylcellulose, carboxymethyl cellulose, aluminum stearate gel or hydrogenated edible fats; emulsifying agents such as lecithin, sorbitan mono-oleate or acacia; non-aqueous vehicles (which may include edible oils) such as almond oil, fractionated coconut oil, oily esters, propylene glycol or ethyl alcohol; and preservatives such as methyl or propyl p-hydroxybenzoates or sorbic acid. Such preparations may also be formulated as suppositories, e.g., containing conventional suppository bases such as cocoa butter or other glycerides.

Pharmaceutical compositions for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The pharmaceutical compositions may be presented in unit-dose or multi-dose containers, for example sealed ampoules and vials, and may be stored in a freeze-dried (lyophilised) condition requiring only the addition of a sterile liquid carrier, for example, water-for-injection, immediately prior to use. Extemporaneous injection solutions and suspensions may be prepared from sterile powders, granules and tablets of the kind previously described.

Pharmaceutical compositions for rectal administration may be presented as a suppository with the usual carriers such as cocoa butter, hard fat or polyethylene glycol.

Pharmaceutical compositions for topical administration in the mouth, for example buccally or sublingually, include lozenges comprising the active ingredient in a flavoured basis such as sucrose and acacia or tragacanth, and pastilles comprising the active ingredient in a basis such as gelatin and glycerin or sucrose and acacia.

The compounds may also be formulated as depot preparations. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

In addition to the ingredients particularly mentioned above, the compositions may include other agents conventional in the art having regard to the type of formulation in question, for example those suitable for oral administration may include flavouring agents.

It will be appreciated by those skilled in the art that reference herein to treatment extends to prophylaxis as well as the treatment of established diseases or symptoms. Moreover, it will be appreciated that the amount of a compound of the invention required for use in treatment will vary with the nature of the condition being treated and the age and the condition of the patient and will be ultimately at the discretion of the attendant physician or veterinarian. In general, however, doses employed for adult human treatment will typically be in the range of 0.02-5000 mg per day, preferably 1-1500 mg per day. The desired dose may conveniently be presented in a single dose or as divided doses administered at appropriate intervals, for example as two, three, four or more sub-doses per day. The formulations according to the invention may contain between 0.1-99% of the active ingredient, conveniently from 30-95% for tablets and capsules and 3-50% for liquid preparations.

The compound of formula (I) for use in the instant invention may be used in combination with other therapeutic agents for example, statins and/or other lipid lowering drugs for example MTP inhibitors and LDLR upregulators. The compounds of the invention may also be used in combination with antidiabetic agents, e.g. metformin, sulfonylureas and/or PPAR gamma agonists (for example thiazolidinediones such as e.g. Pioglitazone and Rosiglitazone). The compounds may also be used in combination with antihypertensive agents such as calcium channel antagonists and ACE inhibitors. The invention thus provides in a further aspect the use of a combination comprising a compound of formula (I) with a further therapeutic agent in the treatment of a hPPAR alpha mediated disease.

When the compounds of formula (I) are used in combination with other therapeutic agents, the compounds may be administered either sequentially or simultaneously by any convenient route.

The combinations referred to above may conveniently be presented for use in the form of a pharmaceutical composition and thus pharmaceutical compositions comprising a combination as defined above optimally together with a pharmaceutically acceptable carrier or excipient comprise a further aspect of the invention. The individual components of such combinations may be administered either sequentially or simultaneously in separate or combined pharmaceutical formulations.

When combined in the same pharmaceutical composition it will be appreciated that the two compounds must be stable and compatible with each other and the other components of the formulation and may be formulated for administration. When formulated separately they may be provided in any convenient pharmaceutical composition, conveniently in such a manner as are known for such compounds in the art.

When a compound of the invention is used in combination with a second therapeutic agent active against the same hPPAR mediated disease, the dose of each compound may differ from that when the compound is used alone. Appropriate doses will be readily appreciated by those skilled in the art.

Compounds of this invention may be conveniently prepared by a general process wherein a moiety like (A) is acylated with an ester (B). R in formula (B) is C₁₋₆alkyl (preferably ethyl). Note this synthesis is preferably carried out with the acid group of moiety A protected by R to avoid decarboxylation of the acid. Thus R may be hydrogen but is conveniently C₁₋₆ alkyl which can be hydrolyzed off to give an acid of Formula (I), or if readily hydrolyzable, the resulting ester can be administered.

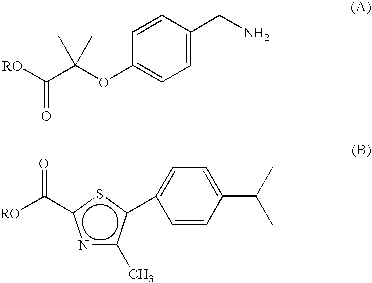

In particular, the compound of formula (I) may be obtained as described in the reaction scheme below.

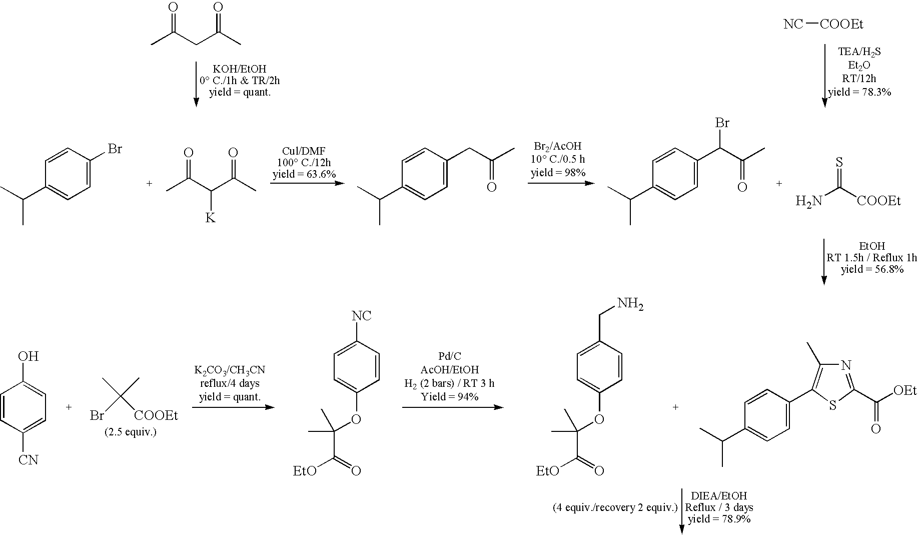

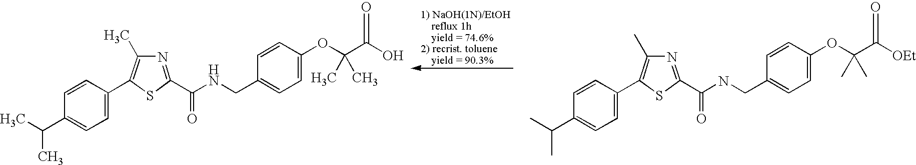

In an alternative route, the coupling of A and B may occur with A being an acid (i.e. R in moiety A is H).

### EXAMPLES

The invention will now be demonstrated by the following intermediates and examples which should not be construed as constituting a limitation thereto. 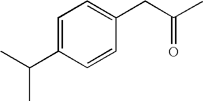
Intermediate 1: 1-[4-(1-methylethyl)phenyl]-2-propanone

513 ml of 2,4-pentanedione was slowly added to a solution of 280.5 g of KOH in 2.3 L of ethanol cooled at 5° C., over 2.5 h,. The mixture was stirred at 5° C. for 1 h then heated to room temperature and stirred for 2 h. The mixture was concentrated to dryness to give 639 g of the potassium salt of 2,4-pentanedione used without further purification. Yield=92.6%.

Under nitrogen and at room temperature, 86.9 g of copper iodide and 630 g of potassium salt of 2,4-pentanedione was added to a solution of 181.8 g of 1-bromo-4-(1-methylethyl)benzene in DMF. The mixture was stirred at 100° C. for 16 h then, after cooling to 10° C., 2 L of H₂O were added. The aqueous layer was extracted 4 times with pentane. The organic layer was washed with HCl 1N and dried on MgSO₄. After filtration and evaporation under vacuum, the oily residue was purified on silicagel (Cyclohexane/AcOEt=95/5) to give after evaporation 76.9 g of a colorless oil. Yield=47.8%.

¹H-NMR (CDCl₃): δ 7.12 (d, 2H); 7.05 (d, 2H); 3.58 (s, 2H); 2.81 (h, 1H); 2.07 (s, 3H); 1.16 (d, 6H). 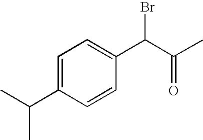
Intermediate 2: 1-bromo-1-[4-(1-methylethyl)phenyl]-2-propanone

a solution of 37.2 ml of bromine in 370 ml of acetic acid was added dropwise over 1 hour to a solution of 130.3 g of Intermediate 1 (1-[4-(1-methylethyl)phenyl]-2-propanone) in 500 ml of acetic acid cooled at 10° C. The mixture was stirred for 10 minutes after the end of the addition then poured into 1.4 L of cold H₂O. The aqueous solution was treated with 70 g of Na₂SO₃ then stirred for 1 h. The aqueous layer was extracted twice with CH₂Cl₂. The organic layer was washed twice with water and dried on MgSO₄. After filtration and evaporation under vacuum , 183 g of a green oil was obtained and used without further purification. Yield=96.9%.

¹H-NMR (CDCl₃): δ 7.28 (d, 2H); 7.16 (d, 2H); 5.35 (s, 1H); 2.84 (h, 1H); 2.23 (s, 3H); 1.17 (d, 6H). 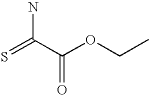
Intermediate 3: Ethyl amino(thioxo)acetate

3 ml g of triethylamine was added to a solution of 100 g of Ethyl cyanoformate in 400 mL of diethyl ether. The reaction mixture was cooled to 0° C. and H₂S was bubbled for 1 h. After the end of addition the mixture was allowed to return to room temperature and stirred overnight. Then the mixture was poured in 900 ml of HCl 1N. After stirring for 0.5 h and decantation, the aqueous layer was extracted twice with 200 ml of diethyl ether. The combined organic layer was washed 3 times with 300 ml of H₂O. After drying on magnesium sulphate, the organic layer was filtered and concentrated to dryness to give 109.5 g of yellow crystals which are used in the next step without further purification. yield=81.6%.

¹H-NMR (CDCl₃): δ 8.2 (b, 1H); 7.7 (b, 1H); 4.3 (q, 2H); 1.34 (t, 3H). 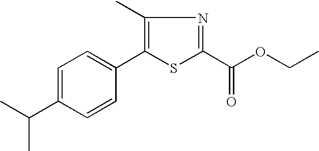
Intermediate 4: Ethyl 4-methyl-5-[4-(1-methylethyl)phenyl]-1.3-thiazole-2-carboxylate

95.4 g of Intermediate 3 (ethyl amino(thioxo)acetate) was added to a solution of 183 9 of Intermediate 2 (1-bromo-1-[4-(1-methylethyl)phenyl]-2-propanone) in 2 L of EtOH. The reaction mixture was stirred overnight at room temperature then heated at reflux for 45 minutes. The mixture was concentrated to dryness and the residue is purified on silicagel (Cyclohexane/AcOEt=9/1 then 8/2) to give after evaporation 108.3 9 of an orange oil. Yield=52.3%.

¹H-NMR (CDCl₃): δ 7.42 (d, 2H); 7.33 (d, 2H); 4.5 (q, 2H); 2.98 (h, 1H); 2.61 (s, 3H; 1.46 (t, 3H); 1.31 (d, 6H). 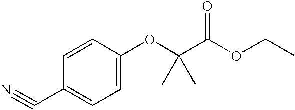
Intermediate 5: Ethyl 2-[(4-cyanophenyl)oxy]-2-methylpropanoate

278.6 g of K₂CO₃ was added to a solution of 200 g para hydroxybenzonitrile in 2 L of acetonitrile. The mixture was refluxed for 1 h, then 250 ml of ethyl bromoisobutyrate was slowly added over 1 hour. The reaction was followed by ttc. After 2 days of reflux, 140 g of K₂CO₃ and 126 ml of ethyl bromoisobutyrate were added. After 6 h of reflux, 140 g of K₂CO3 and 126 ml of ethyl bromoisobutyrate were added. After 3 days of reflux, 140 g of K₂CO₃ and 126 ml of ethyl bromoisobutyrate were added. After 6 h of reflux and return to room temperature, the solid material was removed by filtration and washed with acetonitrile. The filtrate was concentrated to dryness then treated with 800 ml of NaOH 1N. After stirring for 15 minutes, the aqueous mixture was extracted twice with ethyl ether. The organic layer was washed with NaOH 1N and brine. After drying on magnesium sulphate, the organic layer was filtered and concentrated to dryness to give 364 g of an oil. yield=92.9%.

¹H NMR (CDCl₃): δ 7.37 (d, 2H), 6.68 (d, 2H), 4.06 (q, 2H), 1.48 (s, 6H), 1.05 (t, 3H) 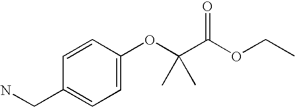
Intermediate 6: Ethyl 2-{[4aminomethyl)phenyl]oxy}-2-methylpropanoate

In a hydrogenator of 1 L, a mixture of 62.3 g of Intermediate 5: (Ethyl 2-[(4-cyanophenyl)oxy]-2-methylpropanoate), 47 ml of glacial acetic acid and 3.1 g of Pd/C 10% in 310 ml of ethyl alcohol was hydrogenated over 2 bars of hydrogen and at room temperature. After filtration of the catalyst, the solution was evaporated to dryness to give the acetic salt of Ethyl 2-{[4-(aminomethyl)phenyl]oxy}-2-methylpropanoate (oily residue).

The residue was poured in 350 ml of water 500 ml of ethyl acetate. The biphasic mixture was cooled to 10° C. and treated with NaOH 1N (to pH=11). After decantation, the aqueous layer was extracted twice with 200 ml of ethyl acetate. The combined organic layer was washed with 300 ml of brine. After drying on magnesium sulphate, the organic layer was filtered and concentrated to dryness to give 60.1 g of a colorless oil crude which is used in the next step without further purification. yield=94.8%.

¹H NMR (CDCl₃): δ 7.07 (d, 2H), 6.71 (d, 2H), 4.12 (q, 2H), 3.68 (m, 2H), 1.47 (s, 6H), 1.15 (t, 3H).

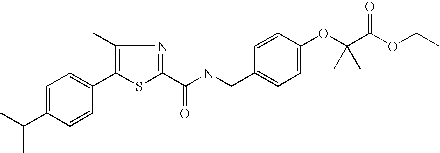

Intermediate 7: Ethyl 2-methyl-2-[(4-{[({4-methyl-5-[4-(1-methylethyl)phenyl]-1,3-thiazol-2-yl}carbonyl)amino]methyl}phenyl)oxy]propanoate

229 g of Intermediate 6 (Ethyl 2-{[4-(aminomethyl)phenyl]oxy}-2-methylpropanoate) and 420 ml of diisopropylethylamine. Were added to a solution of 139.6 g of Intermediate 4 (Ethyl 4-methyl-5-[4-(1-methylethyl)phenyl]-1,3-thiazole-2-carboxylate) in 1.5 L of EtOH. The reaction mixture was heated at reflux for 3 days, then a solution of 1 equiv of Ethyl 2-{[4-(aminomethyl)phenyl]oxy}-2-methylpropanoate in 250 ml of EtOH was added and the reaction mixture was heated at reflux for a further day. This operation was repeated once more. The mixture was concentrated to dryness and 2 L of H₂O was added to the residue then NaOH 1N until a pH=11 was obtained. The aqueous layer was extracted with Ethyl acetate. The organic layer was washed twice with HCl 1N, saturated solution of NaHCO₃ and brine. The organic layer was dried with MgSO₄. After filtration and evaporation under vacuum, the oily residue obtained was purified on silicagel (Cyclohexane/AcOEt=9/1) to give after evaporation 142.9 g of an oil which crystallized slowly. Yield=61.6%.

¹H-NMR (CDCl₃): δ 7.42 (t, 1H); 7.31 (d, 2H); 7.23 (d, 2H); 7.17 (d, 2H); 6.75 (d, 2H); 4.5 (d, 2H); 4.17 (q, 2H); 2.88 (h, 1H); 2.41 (s, 3H); 1.52 (s, 6H); 1.22-1.17 (m, 9H).

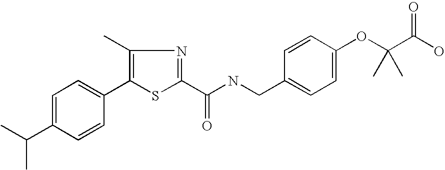

### Example 1

### 2-methyl-2-[(4-{[({4-methyl-5-[4-(1-methylethyl)phenyl]-1,3-thiazol-2-yl}carbonyl)amino]methyl}phenyl)oxy]propanoic acid

was added 595 mL of NaOH 1N was added to a solution of 142.9 g of Intermediate 7 (Ethyl-2-methyl-2-[(4-{[({4-methyl-5-[4-(1-methylethyl)phenyl]-1,3-thiazol-2-yl}carbonyl)amino]methyl}phenyl)oxy]propanoate) in 2 L of EtOH. The reaction mixture was heated at reflux for 1 H. EtOH was evaporated under vacuum and 1 L of H2O was added then HCl 1N until pH=1 was obtained. A white precipitate appeared and the mixture was stirred at 5° C. for 15 minutes to obtain complete precipitation. The white precipitate was filtered then washed with water. After drying under vacuum the white powder obtained was re-crystallised in toluene (1 g of compound in 1.7 ml of toluene) to afford 88.2 g of off white crystals. Yield=65.5%. Mp=130.8° C.

¹H NMR (CDCl₃): δ 7.86 (t, 1H); 7.39 (d, 2H); 7.33-7.28 (m, 4H); 6.93 (d, 2H); 4.6 (d, 2H); 2.98 (h, 1H); 2.5 (s, 3H); 1.63 (s, 6H); 1.31 (d, 6H).

Binding and Transfection Assays

Binding Assay:

Compounds were tested for their ability to bind to hPPAR gamma hPPARalpha or PPARdelta using a Scintillation Proximity Assay (SPA). The PPAR ligand binding domain (LBD) was expressed in E. coli as polyHis tagged fusion proteins and purified. The LBD was then labelled with biotin and immobilised on streptavidin-modified scintillation proximity beads. The beads were then incubated with a constant amount of the appropriate radioligand (5-{4-[2-Methyl-pyridin-2-yl-amino)-ethoxy]-benzyl}-thiazolidine-2,4-dione (J. Med. Chem. 1994, 37(23), 3977), for PPARgamma), and labelled GW 2433 (see Brown, P. J et al . Chem. Biol., 4, 909-918 (1997), for the structure and synthesis of this ligand) for PPAR alpha and PPAR delta) and variable concentrations of test compound, and after equilibration the radioactivity bound to the beads was measured by a scintillation counter. The amount of nonspecific binding, as assessed by control wells containing 50 μM of the corresponding unlabeled ligand, was subtracted from each data point. For each compound tested, plots of ligand concentration vs. CPM of radioligand bound were constructed and apparent KI values were estimated from nonlinear least squares fit of the data assuming simple competitive binding. The details of this assay have been reported elsewhere (see, Blanchard, S. G. et. al. Development of a Scintillation Proximity Assay for Peroxisome Proliferator-Activated Receptor gamma Ligand Binding Domain. Anal. Biochem., 257, 112-119 (1998)).

Transfection assay:

Compounds were screened for functional potency in transient transfection assays in CV-1 cells for their ability to activate the PPAR subtypes (transactivation assay). A previously established chimeric receptor system was utilized to allow comparison of the relative transcriptional activity of the receptor subtypes on the same target gene and to prevent endogenous receptor activation from complicating the interpretation of results. See, for example, Lehmann, J. M.; Moore, L. B.; Smith-Oliver, T. A.; Wilkison, W. O.; Willson, T. M.; Kliewer, S. A., An antidiabetic thiazolidinedione is a high affinity ligand for peroxisome proliferator-activated receptor gamma (PPARgamma), J. Biol. Chem., 270, 12953-6 (1995). The ligand binding domains for murine and human PPAR alpha, PPAR gamma, and PPAR delta were each fused to the yeast transcription factor GAL4 DNA binding domain. CV-1 cells were transiently transfected with expression vectors for the respective PPAR chimera along with a reporter construct containing five copies of the GAL4 DNA binding site driving expression of secreted placental alkaline phosphatase (SPAP) and beta-galactosidase. After 16 h, the medium was exchanged to DME medium supplemented with 10% delipidated fetal calf serum and the test compound at the appropriate concentration. After an additional 24 h, cell extracts were prepared and assayed for alkaline phosphatase and β-galactosidase activity. Alkaline phosphatase activity was corrected for transfection efficiency using the beta-galactosidase activity as an internal standard (see, for example, Kliewer, S. A., et. al. Cell 83, 813-819 (1995)). Rosiglitazone (BRL 49653) was used as a positive control in the hPPAR gamma assay. The positive control in the hPPAR alpha assays was 2-4-[2-(3-[4-fluorophenyl]-1-heptylureido)ethyl]-phenoxy-2-methyl propionic acid (WO 97/36579). The positive control for PPAR delta assays was 2-{2-methyl-4-[({4-methyl-2-{trifluoromethyl)phenyl]-1,3-thiazol-5-yl}methyl)sulfanyl]phenoxy}acetic acid (WO 01/00603). The positive control was (5-{4-[2-Methyl-pyridin-2-yl-amino)-ethoxy]-benzyl}-thiazolidine-2,4-dione (J. Med. Chem. 1994, 37(23), 3977), for PPAR gamma.

Activities in three hPPAR subtypes are reported in the table and are expressed in micromolar.

| | EC50 μM | EC50 μM | EC50 μM |
|---|---|---|---|
| Example | HPPARα | HPPARδ | HPPARγ |
| Example 1 | 0.008 | 6.1 | 0.19 |

What is claimed is:

1. A compound of formula (I) 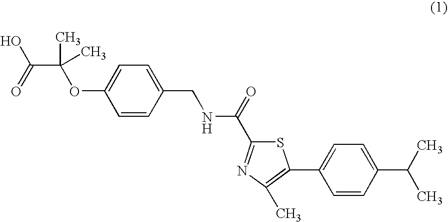
and pharmaceutically acceptable salts, solvates and hydrolysable esters thereof.

2. (canceled)

3. (canceled)

4. A pharmaceutical composition comprising a compound according to claim 1 and a pharmaceutically acceptable diluent or carrier.

5. (canceled)

6. (canceled)

7. A method of treating a hPPAR mediated disease or condition in a patient comprising the administration of a compound according to claim 1.

8. A method according to claim 7 wherein the hPPAR mediated disease or condition is dyslipidemia, syndrome X, heart failure, hypercholesteremia, cardiovascular disease, type II diabetes mellitus, type I diabetes, insulin resistance, hyperlipidemia, obesity, anorexia bulimia or anorexia nervosa.
